(19) United States
(12) Patent Application Publication
(10) Pub. No.: US 2023/0173466 A1
(43) Pub. Date: Jun. 8, 2023

(54) METHODS OF PRODUCING CATALYSTS FOR VINYL ACETATE PRODUCTION

(71) Applicant: CELANESE INTERNATIONAL CORPORATION, Irving, TX (US)

(72) Inventors: Steve R. ALEXANDER, Houston, TX (US); Justin DODSON, League City, TX (US); Laiyuan CHEN, Houston, TX (US)

(73) Assignee: CELANESE INTERNATIONAL CORPORATION, Irving, TX (US)

(21) Appl. No.: 17/997,011

(22) PCT Filed: Jun. 8, 2021
(86) PCT No.: PCT/US2021/036344

§ 371 Date: Oct. 24, 2022

Related U.S. Application Data
(60) Provisional application No. 63/037,657, filed on Jun. 11, 2020.

(51) Int. Cl. B01J 23/66; B01J 37/18; C07C 69/15

(52) U.S. Cl. CPC B01J 23/66; B01J 37/18; C07C 69/15; B01J 2523/824; C07C 2523/52; C07C 2523/44

(57) ABSTRACT

Methods of producing gold-palladium catalysts suitable for use in the production of vinyl acetate may include drying the catalyst after the incorporation of a promoter at higher temperatures (e.g., 160° C. or greater) to restructure the metals and/or alloys on the catalyst. The restructured catalyst advantageously has increased catalytic activity and improved stability.

### BACKGROUND

Vinyl acetate is produced by reacting ethylene, oxygen, and acetic acid in the presence of a catalyst (e.g., palladium and/or gold supported on a carrier). Further, the inclusion of compounds like sodium acetate, potassium acetate, and cesium acetate have been shown to increase the yield and selectivity of the reaction to vinyl acetate. Said acetates may be impregnated on the support and/or introduced with the feed to the reactor.

### BRIEF DESCRIPTION OF THE DRAWINGS

The following figures are included to illustrate certain aspects of the disclosure, and should not be viewed as exclusive configurations. The subject matter disclosed is capable of considerable modifications, alterations, combinations, and equivalents in form and function, as will occur to those skilled in the art and having the benefit of this disclosure.

FIG. 1 illustrates a flow diagram of a nonlimiting example method for the preparation of catalysts described herein.

FIG. 2 illustrates a process flow diagram of an example vinyl acetate production process of the present disclosure.

FIG. 3 is the x-ray diffraction (XRD) data for catalyst samples dried at 100° C., 140° C., or 180° C.

### DETAILED DESCRIPTION

The present disclosure relates to methods of producing catalysts suitable for use in the production of vinyl acetate. More specifically, the methods described herein include higher drying temperatures after the incorporation of a promoter. Without being limited by theory, it is believed that heating to 160° C. or more after impregnation with the promotor changes the structure of the catalyst. The restructured catalyst advantageously has increased catalytic activity and improved stability, which reduces the time for oxygen break-in. Without being limited by theory, it is believed that the restructuring of the catalyst relates to the PdAu alloy composition restructuring to a more thermodynamically favored PdAu alloy.

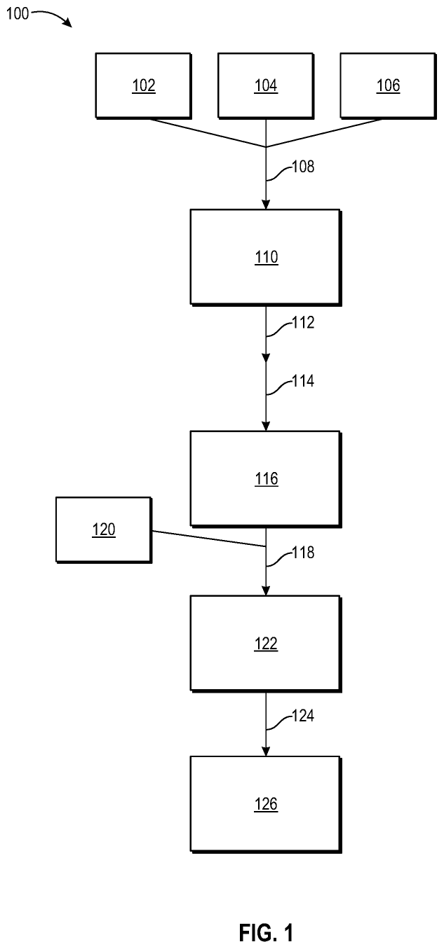

FIG. 1 illustrates a flow diagram of a nonlimiting example method for the preparation of catalysts described herein. Generally, methods of the present disclosure include: impregnating 108 a porous support 102 with a water-insoluble gold compound and a water-insoluble palladium compound to yield a precipitated support 110 by precipitating a water-soluble gold compound 104 and a water-soluble palladium compound 106 in the presence of the porous support 102; washing 112 the precipitated support 110; reducing 114 the water-insoluble gold compound and the water-insoluble palladium compound on the precipitated support 110 to yield a metal-impregnated support 116; impregnating 118 the metal-impregnated support 116 with an alkali metal promoter 120 to yield a metal/promoter-impregnated support 122; and drying 124 the metal/promoter-impregnated support 122 at 160° C. or greater to yield a catalyst 126.

The impregnation 108 of the porous support 102 with the water-insoluble gold compound 104 and the water-insoluble palladium compound 106 may be performed simultaneously by (a) mixing (or impregnating) the porous support 102 with an aqueous solution of the water-soluble gold compound 104 and the water-soluble palladium compound 106 and, then, (b) adding a precipitation agent to the mixture so as to precipitate the water-soluble gold compound 104 and the water-soluble palladium compound 106 as the water-insoluble gold compound and the water-insoluble palladium compound, respectively, onto the porous support 102. Alternatively, the water-insoluble gold compound 104 and the water-insoluble palladium compound 106 may be precipitated in separate steps. For example, impregnation 108 may comprise: (a) mixing (or impregnating) the porous support 102 with an aqueous solution of the water-soluble palladium compound 106, (b) adding a precipitation agent to the mixture so as to precipitate the water-soluble palladium compound 106 as the water-insoluble palladium compound onto the porous support 102, (c) washing the porous support having the water-insoluble palladium compound thereon, (d) mixing (or impregnating) the porous support having the water-insoluble palladium compound thereon with an aqueous solution of the water-soluble gold compound 104, and (e) adding a precipitation agent (the same or different precipitating agent as used for the water-soluble palladium compound 106) to the mixture so as to precipitate the water-soluble gold compound 104 as the water-insoluble gold compound to yield the precipitated support 110. Alternatively, impregnation 108 may comprise: (a) mixing (or impregnating) the porous support 102 with an aqueous solution of the water-soluble gold compound 104, (b) adding a precipitation agent to the mixture so as to precipitate the water-soluble gold compound 104 as the water-insoluble gold compound onto the porous support 102, (c) washing the porous support having the water-insoluble gold compound thereon, (d) mixing (or impregnating) the porous support having the water-insoluble gold compound thereon with an aqueous solution of the water-soluble palladium compound 106, and (e) adding a precipitation agent (the same or different precipitating agent as used for the water-soluble gold compound 104) to the mixture so as to precipitate the water-soluble palladium compound 106 as the water-insoluble palladium compound to yield the precipitated support 110.

The porous support 102 may be of any diverse geometrical shape. For example, the shapes of the porous supports 102 may include, but are not limited to, spheres, tablets, cylinders, fibers, facetted particles, and the like, and any hybrid thereof. Preferably, the porous support 102 has a diameter of about 1 mm to about 10 mm (or about 1 mm to about 5 mm, or about 3 mm to about 8mm, or about 5 mm to about 10 mm). The diameter of the porous supports 102 can be measured by light scattering techniques or microscopy. Preferably, the porous supports 102 are spherical with diameters of about 4 mm to about 8 mm. It would be recognized by one skilled in the art that the shape of the porous supports 102 will likely vary from a precise shape described. For example, the porous supports 102 described as spherical have a generally spherical shape.

The surface area of the porous support 102 may be about 10 m²/g to about 350 m²/g (or about 10 m²/g to about 150 m²/g, or about 100 m²/g to about 200 m²/g, or about 150 m²/g to about 350 m²/g). The pore volume of the porous support 102 may be about 0.1 cm³/g to about 2 cm³/g (about 0.1 cm³/g to about 1 cm³/g, or about 0.5 cm³/g to about 1.5 cm³/g, or about 1 cm³/g to about 2 cm³/g). The surface area and pore volume can be measured and/or derived from measurements according to BET nitrogen adsorption per ASTM D5601-96(2017).

Examples of porous supports 102 include, but are not limited to, silica, alumina, aluminum silicates, titania, zirconia, spinels, carbon, and the like, and any combination thereof. Silica is the preferred porous support 102.

Examples of water-insoluble gold compounds 104 include, but are not limited to, auric (III) chloride, tetrahaloauric (III) acid, and the like, and any combination thereof.

Examples of water-soluble palladium compounds 106 include, but are not limited to, palladium (II) chloride, sodium palladium (II) chloride, alkali earth metal tetrachloropalladium (II), palladium (II) nitrate, palladium (II) sulfate, and the like, and any combination thereof.

Generally, the gold is present at a lower molar concentration than the palladium. A molar ratio of gold to palladium on the precipitated support 110 (and consequently the metal-impregnated support 116, the metal/promoter-impregnated support 122, and the catalyst 126) may be about 0.01:1 to about 0.7:1 (or about 0.01:1 to about 0.1:1, or about 0.1:1 to about 0.5:1, or about 0.3:1 to about 0.7:1).

The total amount of metal (as the gold and palladium not the salt) on the precipitated support 110 (and consequently the metal-impregnated support 116, the metal/promoter-impregnated support 122, and the catalyst 126) may be about 0.05 wt % to about 20 wt % (or about 0.05 wt % to about 10 wt %, or about 1 wt % to about 15 wt %, or about 5 wt % to about 20 wt %)

The amount of time and temperature that the porous support 102 is exposed to the water-soluble metal salts 104 and 106 before precipitation may vary. The time may range from about 10 minutes to about 2 days (or about 30 minutes to about 1 day, or about 1 hour to about 6 hours). The temperature may range from about 20° C. to about 50° C. (or about 23° C. to about 40° C.).

The mixing (or impregnating) of the porous support 102 with the water-soluble metal salts 104 and 106 may be by mixing the components together, with optional heating, and allowing time to pass with or without additional or continued mixing. Further, while impregnating, the water of the aqueous solution of the water-soluble metal salts 104 and 106 may optionally be allowed to evaporate such that the remaining mixture is 10 wt % or less (or 5 wt % or less, or 1 wt % or less) water. Various rotation, tumbling, or equivalent equipment may be used for the mixing (or impregnating) steps.

Examples of precipitating agents include, but are not limited to, alkali metal hydroxides, alkali metal bicarbonates and/or alkali metal carbonates, alkali metal silicates, alkali metal borates, hydrazine, and the like, and any combination thereof. Preferably, the precipitating agent is sodium hydroxide and/or potassium hydroxide where the water-insoluble salts of gold and/or palladium may be hydroxides and/or oxides. The precipitating agents are typically in an aqueous solution. The amount of the precipitating agents should be sufficient to ensure that all of the palladium and gold water-soluble salts are precipitated in the form of water-insoluble salts. To ensure suitable precipitation, the amount of precipitating agents present is preferably approximately 1 to 3 times (or 1.1 to 2 times) the amount of total anions present in the water-soluble metal salts.

Washing after precipitation may be performed with water (e.g., deionized water) or other suitable solvent that does not solubilize the water-insoluble metal salts but does solubilize the anion (e.g., chloride) resulting from the precipitation process. Preferably, washing is performed until about 1000 ppm or less of said anion is present in the wash effluent.

After washing 112 the precipitated support 110, the precipitated support 110 is exposed to a reducing agent. Between washing 112 and reducing 114, the precipitated support 110 may be dried (e.g., in an inert atmosphere like nitrogen, argon, or air at temperatures of about 50° C. to about 150° C. for about 30 minutes to about 3 days).

Reducing 114 may be performed in a liquid phase or in a gas phase. For example, reducing 114 in the liquid phase may be performed using aqueous hydrazine hydrate. Said liquid phase methods may be performed at temperatures of about 20° C. to about 50° C. (or about 23° C. to about 30° C.) for a time sufficient (e.g., about 1 hour to about 24 hours) to convert at least 95 mol % (or at least 98 mol %) of the insoluble-metal salts to metals.

Reducing 114 in the gas phase may be performed using, for example, hydrogen and/or hydrocarbons (e.g., ethylene). Optionally, an inert carrier gas like nitrogen or argon may be utilized in gas phase methods where, for example, the concentration of the hydrogen and/or hydrocarbons is cumulatively about 0.1 vol % to about 10 vol % (or about 0.5 vol % to about 5 vol %) of the gas to which the precipitated supports 110 are exposed. The gas phase reducing methods may be performed at temperatures of about 50° C. to about 250° C. (or about 100° C. to about 200° C.) for a time (e.g., about 1 hour to about 24 hours) sufficient to convert at least 95 mol % (preferably at least 98 mol %) of the insoluble-metal salts to metals.

Reducing 114 yields the metal-impregnated support 116, which is then impregnated 118 with the alkali metal promoter 120 to yield a metal/promoter-impregnated support 122. Examples of alkali metal promoters 120 include, but are not limited to, a sodium salt, a potassium salt, or a cesium salt of formic acid, acetic acid, propionic acid, butyric acid, and the like, and any combination thereof. Potassium metal promoters are preferred. Potassium acetate is the preferred alkali metal promoter 120.

The amount of time and temperature that the metal-impregnated support 116 is exposed to the alkali metal promoter 120 may vary. The time may range from about 1 minute to about 6 hours (or about 1 minute to about 1 hour, or about 30 minutes to about 1 day, or about 1 hour to about 6 hours). The temperature may range from about 20° C. to about 50° C. (or about 23° C. to about 40° C.).

The mixing (or impregnating) of the metal-impregnated support 116 with the alkali metal promoter 120 may be by mixing the components together, with optional heating, and allowing time to pass with or without additional or continued mixing. Further, while impregnating, the water of the alkali metal promoter 120 may optionally be allowed to evaporate such that the remaining mixture is 10 wt % or less (or 5 wt % or less, or 1 wt % or less) water. Various rotation, tumbling, or equivalent equipment may be used for the mixing (or impregnating) steps.

The metal/promoter-impregnated support 122 is then dried 124 at 160° C. or greater (about 160° C. to about 250° C., or about 160° C. to about 200° C., or about 200° C. to about 250° C.) to yield a catalyst 126. Again, without being limited by theory, it is believed that temperatures greater than 160° C. will restructure the catalyst (as illustrated by the XRD data having a lower 20 value at the peak intensity between a 20 of 38° and)40°. Further, it is believed that temperatures higher than 250° C. will cause sintering of the catalyst and, consequently, begin deactivating the catalyst. Preferably, the catalyst 126 has a 20 value for the peak XRD intensity between 38° and 40° of about 38.6° to about 39.2° (or about 38.7° to about 39.1°, or about 38.8° to about)39.1°. XRD is performed using a powder sample that is loaded into an in-situ cell. XRD measurements, unless otherwise specified, are performed in an atmosphere of nitrogen or air and at 25° C.

Drying 124 may be in an inert atmosphere like nitrogen, argon, or air for about 10 minutes to about 1 day (or about 10 minutes to about 3 hours, or about 30 minutes to about 8 hours, or about 6 hours to about 1 day). Drying 124 may be in any suitable system including, but not limited to, a fluid bed dryer, a belt dryer, or any other drying vessel.

The alkali metal promoter 120 may be present at about 0.1 wt % to about 10 wt % (about 0.1 wt % to about 5 wt %, or about 1 wt % to about 7 wt %, or about 5 wt % to about 10 wt %) of the catalyst 126 on a dry basis.

Accordingly, a catalyst of the present disclosure may comprise: (a) gold, palladium, and/or gold-palladium alloy, (b) an alkali metal promoter, and (c) a porous support, wherein the catalyst has a 20 value for a peak x-ray diffraction intensity between 38° and 40° of about 38.6° to about 39.2°.

The catalyst of the present disclosure may be used in the synthesis of vinyl acetate from ethylene, oxygen, and acetic acid in the gas phase. For example, a method may comprise: reacting ethylene, oxygen, and acetic acid in the presence of a catalyst of the present disclosure to produce vinyl acetate.

The catalysts prepared by the methods described herein may be used in a variety of vinyl acetate synthesis methods and systems including fluidized bed reactor, gas phase reactor, or stirred tank reactor methods and systems. Examples of vinyl acetate synthesis methods and systems are described in U.S. Pat. Nos. 5,731,457, 5,968,860, 6,107,514, 6,420,595, 8,822,717 and US Patent App. Pub. No. 2010/0125148, each of which is incorporated herein by reference.

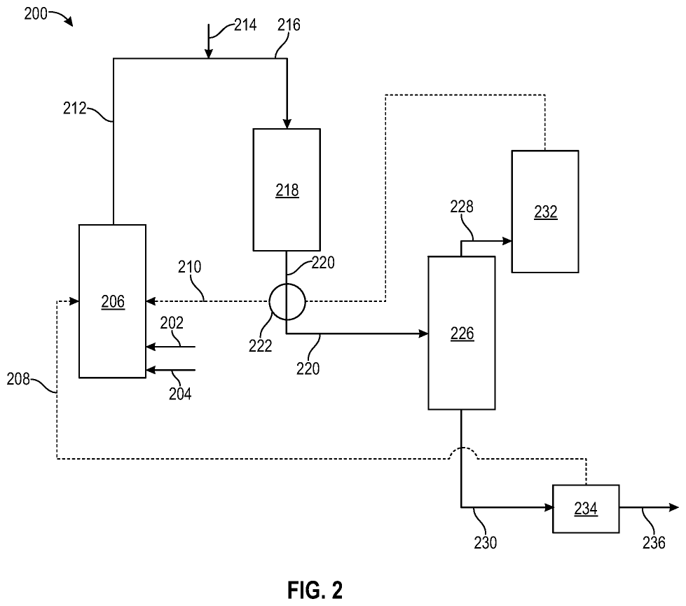

By way of nonlimiting example, FIG. 2 illustrates a process flow diagram of an example vinyl acetate production process 200 in which the catalyst of the present disclosure may be implemented. Additional components and modifications may be made to the process 200 without changing the scope of the present invention. Further, as would be recognized by one skilled in the art, the description of the process 200 and related system uses streams to describe the fluids passing through various lines. For each stream, the related system has corresponding lines (e.g., pipes or other pathways through which the corresponding fluids or other materials may pass readily) and optionally valves, pumps, compressors, heat exchangers, or other equipment to ensure proper operation of the system whether explicitly described or not.

Further, the descriptor used for individual streams does not limit the composition of said streams to consisting of said descriptor. For example, an ethylene stream does not necessarily consist of only ethylene. Rather, the ethylene stream may comprise ethylene and a diluent gas (e.g., an inert gas). Alternatively, the ethylene stream may consist of only ethylene. Alternatively, the ethylene stream may comprise ethylene, another reactant, and optionally an inert component.

In the illustrated process 200, an acetic acid stream 202 and an ethylene stream 204 are introduced to a vaporizer 206. Optionally, ethane may also be added to the vaporizer 206. In addition, one or more recycle streams (illustrated as recycle streams 208 and 210) may also be introduced to the vaporizer 206. While the recycle streams 208 and 210 are illustrated as being directly introduced to the vaporizer 206, said recycle streams or other recycle streams may be combined (not shown) with the acetic acid stream 202 before introduction to the vaporizer 206.

The temperature and pressure of vaporizer 206 may vary over a wide range. The vaporizer 206 preferably operates at a temperature from 100° C. to 250° C., or from 100° C. to 200° C., or from 120° C. to 150° C. The operating pressure of the vaporizer 206 preferably is from 0.1 MPa to 2 MPa, or 0.25 MPa to 1.75 MPa, or 0.5 MPa to 1.5 MPa. The vaporizer 206 produces a vaporized feed stream 212. The vaporized feed stream 212 exits the vaporizer 206 and combines with an oxygen stream 214 to produce a combined feed stream 216 prior to being fed to a vinyl acetate reactor 218.

Regarding the general operating conditions of the vinyl acetate reactor 218, the molar ratio of ethylene to oxygen when producing vinyl acetate is preferably less than 20:1 in the vinyl acetate reactor 216 (e.g., 1:1 to 20:1, or 1:1 to 10:1, or 1.5:1 to 5:1, or 2:1 to 4:1). Further, the molar ratio of acetic acid to oxygen is preferably less than 10:1 in the vinyl acetate reactor 216 (e.g., 0.5:1 to 10:1, 0.5:1 to 5:1, or 0.5:1 to 3:1). The molar ratio of ethylene to acetic acid is preferably less than 10:1 in the vinyl acetate reactor 218 (e.g., 1:1 to 10:1, or 1:1 to 5:1, or 2:1 to 3:1). Accordingly, the combined feed stream 216 may comprise the ethylene, oxygen, and acetic acid in said molar ratios.

The vinyl acetate reactor 218 may be a shell and tube reactor that is capable, through a heat exchange medium, of absorbing heat generated by the exothermic reaction and controlling the temperature therein within a temperature range of 100° C. to 250° C., or 110° C. to 200° C., or 120° C. to 180° C. The pressure in the vinyl acetate reactor 218 may be maintained at 0.5 MPa to 2.5 MPa, or 0.5 MPa to 2 MPa.

Further, the vinyl acetate reactor 218 may be a fixed bed reactor or a fluidized bed reactor, preferably a fixed bed reactor that contains a catalyst prepared by method of the present disclosure.

The vinyl acetate reaction in the reactor 218 produces a crude vinyl acetate stream 220. Depending on conversion and reaction conditions, the crude vinyl acetate stream 220 can comprise 5 wt % to 30 wt % vinyl acetate, 5 wt % to 40 wt % acetic acid, 0.1 wt % to 10 wt % water, 10 wt % to 80 wt % ethylene, 1 wt % to 40 wt % carbon dioxide, 0.1 wt % to 50 wt % alkanes (e.g., methane, ethane, or mixtures thereof), and 0.1 wt % to 15 wt % oxygen. Optionally, the crude vinyl acetate stream 220 may also comprise 0.01 wt % to 10 wt % ethyl acetate. The crude vinyl acetate stream 220 may comprise other compounds such as methyl acetate, acetaldehyde, acrolein, propane, and inerts such as nitrogen or argon. Generally, these other compounds, except for inerts, are present in very low amounts.

The crude vinyl acetate stream 220 passes through a heat exchanger 222 to reduce the temperature of the crude vinyl acetate stream 220. Preferably, the crude vinyl acetate stream 220 is cooled to a temperature of 80° C. to 145° C., or 90° C. to 135° C.

The systems and methods described herein measure the concentration of one or more metal components in the crude vinyl acetate stream 220 or a stream downstream thereof. As described above, the concentration of the metal components can be used to assess, among other things, the health of the system and/or the health of the catalysts.

The crude vinyl acetate stream 220 may then be conveyed to a separator 226 (e.g., a distillation column). Preferably, little to no condensation of the liquefiable components occurs and the cooled crude vinyl acetate stream 220 (post heat exchanger 222) is introduced to the separator 226 as gas.

The energy to separate the components of the crude vinyl acetate stream 220 may be provided by the heat of reaction in the reactor 218. In some embodiments, there may be an optional reboiler (not illustrated) dedicated to increasing the separation energy within the separator 226.

The separator 226 separates the crude vinyl acetate stream 220 into at least two streams: an overheads stream 228 and a bottoms stream 230. The overheads stream 228 can comprise ethylene, carbon dioxide, water, alkanes (e.g., methane, ethane, propane, or mixtures thereof), oxygen, and vinyl acetate. The bottoms stream 230 can comprise vinyl acetate, acetic acid, water, and potentially ethylene, carbon dioxide, and alkanes.

The overheads stream 228 may be further processed 232 (e.g., undergo further separations and/or be augmented with gases like ethylene and/or methane) to eventually produce the recycle stream 210. Again, the use of recycle stream 210 as a feed for the vaporizer 206 (either as-is or previously mixed with another stream) is optional.

The bottoms stream 230 may be further processed 234 (e.g., undergo further purifications and separations) to eventually produce a vinyl acetate product stream 236 and the recycle stream 208. Again, the use of recycle stream 208 as a feed for the vaporizer 206 (either as-is or previously mixed with another stream) is optional.

### Example Embodiments

A first nonlimiting example embodiment of the present disclosure is a method comprising: impregnating a porous support with a water-insoluble gold compound and a water-insoluble palladium compound to yield a precipitated support by precipitating a water-soluble gold compound and a water-soluble palladium compound in the presence of the porous support; washing the precipitated support; reducing the water-insoluble gold compound and the water-insoluble palladium compound on the precipitated support to yield a metal-impregnated support; impregnating the metal-impregnated support with an alkali metal promoter to yield a metal/promoter-impregnated support; and drying the metal/promoter-impregnated support at 160° C. or greater to yield a catalyst. The first nonlimiting example embodiment may further include one or more of: Element 1: wherein the impregnating of the porous support is performed in multiple steps that comprising: impregnating the porous support with a first water-soluble compound that is the water-insoluble gold compound or the water-insoluble palladium compound; precipitating the first water-soluble compound in the presence of the porous support; impregnating the porous support with a second water-soluble compound that is the water-insoluble gold compound or the water-insoluble palladium compound, wherein the first and second water-soluble compounds are different; and precipitating the second water-soluble compound in the presence of the porous support; Element 2: wherein a molar ratio of the gold to the palladium in the catalyst is about 0.01:1 to about 0.7:1; Element 3: wherein an alkali metal of the alkali metal promoter is present at about 0.1 wt % to about 10 wt % of the catalyst on a dry basis; Element 4: wherein the alkali metal promoter is selected from the group consisting of a sodium salt, a potassium salt, or a cesium salt of formic acid, acetic acid, propionic acid, butyric acid, and any combination thereof; Element 5: wherein the drying of the metal/promoter-impregnated support is in the presence of gas comprising nitrogen, argon, and/or air; Element 6: wherein the catalyst has a 20 value for a peak x-ray diffraction intensity between 38° and 40° of about 38.6° to about 39.2°; Element 7: wherein the reducing is in a gas phase comprising hydrogen and/or a hydrocarbon; Element 8: Element 7 and wherein the hydrocarbon is ethylene; Element 9: Element 7 and wherein the gas phase further comprises an inert carrier gas; Element 10: Element 7 and wherein the reducing is at about 50° C. to about 250° C. for about 1 hour to about 24 hours; Element 11: wherein the reducing is performed in a liquid phase using hydrazine hydrate; Element 12: Element 11 and wherein the reducing is at about 20° C. to about 50° C. for about 1 hour to about 24 hours; Element 13: wherein the drying of the metal/promoter-impregnated support is at about 160° C. to about 250° C.; and Element 14: Element 13 and wherein the drying of the metal/promoter-impregnated support is for about 10 minutes to about 1 day. Example of combinations include, but are not limited to, Element 11 (and optionally Element 12) in combination with one or more of Elements 1-10; Element 13 (and optionally Element 14) in combination with one or more of Elements 1-10; Element 7 in combination with one or more of Elements 8-10; Element 1 in combination with one or more of Elements 2-10; Element 2 in combination with one or more of Elements 3-10; Element 3 in combination with one or more of Elements 4-10; Element 4 in combination with one or more of Elements 5-10; and Element 5 in combination with one or more of Elements 6-10.

A second nonlimiting example embodiment is a catalyst produced by the method of the first nonlimiting example embodiment (optionally including one or more of Elements 1-14).

A third nonlimiting example embodiment is a catalyst comprising: (a) gold, palladium, and/or gold-palladium alloy, (b) an alkali metal promoter, and (c) a porous support, wherein the catalyst has a 20 value for a peak x-ray diffraction intensity between 38° and 40° of about 38.6° to about 39.2°. The third nonlimiting example embodiment may further include one or more of: Element 15: wherein a molar ratio of the gold to the palladium, cumulatively as elemental metals and alloy, in the catalyst is about 0.01:1 to about 0.7:1; Element 16: wherein an alkali metal of the alkali metal promoter is present at about 0.1 wt % to about 10 wt % of the catalyst on a dry basis; Element 17: wherein the alkali metal promoter is selected from the group consisting of a sodium salt, a potassium salt, or a cesium salt of formic acid, acetic acid, propionic acid, butyric acid, and any combination thereof and Element 18: wherein the porous support is selected from the group consisting of: silica, alumina, aluminum silicates, titania, zirconia, spinels, carbon, and any combination thereof. Example of combinations include, but are not limited to, Element 15 in combination with one or more of Elements 16-18; Element 16 in combination with one or both of Elements 17-18; and Elements 17 and 18 in combination.

A fourth nonlimiting example embodiment is a method comprising: reacting ethylene, oxygen, and acetic acid in the presence of a catalyst of the present disclosure to produce vinyl acetate. The fourth nonlimiting example embodiment may further include one or more of: Element 19: wherein a molar ratio of ethylene to oxygen is less than about 20:1; Element 20: wherein a molar ratio of acetic acid to oxygen is less than about 10:1; Element 21: wherein a molar ratio of ethylene to acetic acid is less than about 10:1; Element 22: wherein reacting is at about 100° C. to about 250° C.; Element 23: wherein reacting is at about 0.5 MPa to about 2.5 MPa; Element 24: wherein reacting produces 5 wt % to 30 wt % vinyl acetate, 5 wt % to 40 wt % acetic acid, 0.1 wt % to 10 wt % water, 10 wt % to 80 wt % ethylene, 1 wt % to 40 wt % carbon dioxide, 0.1 wt % to 50 wt % alkanes, 0.1 wt % to 15 wt % oxygen, and optionally 0.01 wt % to 10 wt % ethyl acetate; and Element 25: Element 24 and the method further comprising: separating at least a portion of the vinyl acetate from the other products. Examples of combinations include, but are not limited to, two or more of Elements 19-21 in combination; Element 22 and 23 in combination; Element 22 and/or Element 23 in combination with one or more of Elements 19-21; and Element 24 (and optionally Element 25) in combination with one or more of Elements 19-23.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the incarnations of the present inventions. At the very least, and not as an attempt to limit the application of the doctrine of equivalents to the scope of the claim, each numerical parameter should at least be construed in light of the number of reported significant digits and by applying ordinary rounding techniques.

One or more illustrative incarnations incorporating one or more invention elements are presented herein. Not all features of a physical implementation are described or shown in this application for the sake of clarity. It is understood that in the development of a physical embodiment incorporating one or more elements of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having the benefit of this disclosure.

While compositions and methods are described herein in terms of “comprising” various components or steps, the compositions and methods can also “consist essentially of” or “consist of” the various components and steps.

To facilitate a better understanding of the embodiments of the present invention, the following examples of preferred or representative embodiments are given. In no way should the following examples be read to limit, or to define, the scope of the invention.

### EXAMPLES

Catalyst samples were prepared by preparing a masterbatch of Pd/Au/KOAc-impregnated KA-160 using Na₂PdCl₄ and NaAuCl₄ as the water-soluble metal salts, NaOH as the precipitating agent, KA-160 (a silica/alumina support material, available from Sud Chemie) as the porous support, and potassium acetate (KOAc) as the alkali metal promoter. After impregnation with the KOAc, the samples were loaded into an in-situ cell for XRD analysis. Under a nitrogen-containing atmosphere the samples in the XRD were ramped to a specified drying temperature of 100° C., 140° C., or 180° C. The XRD data was collected continuously with a Cu K-alpha source for 20 between 36° and 52° with step size 0.06°. The phases were determined by peak matching to a crystallographic database such as ICDD database.

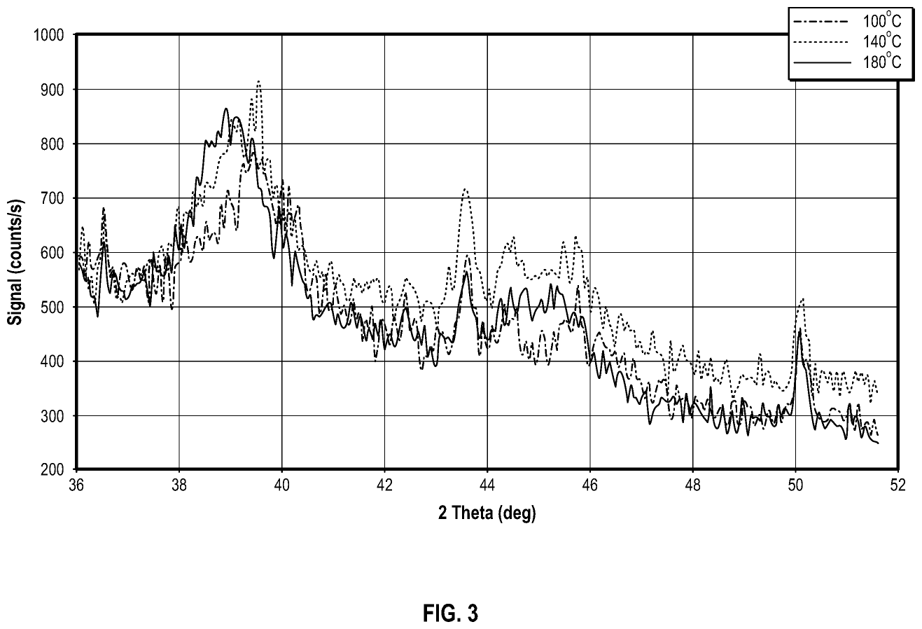

FIG. 3 is the XRD data for the samples at 100° C., 140° C., or 180° C. The XRD spectra show increasing the temperature changes the structure of the catalyst because the peak between 38° and 40° is at a lower 20 value for the 180° C. as compared to the 100° C. and 140° C. samples. More specifically, the 20 value corresponding to the maximum signal between 38° and 40° is 39.4°, 39.5°, and 38.9° for the 100° C., 140° C., or 180° C. samples, respectively. The 20 value corresponding to the maximum signal between 38° and 40° is maintained after cooling to room temperature. Further, such 20 value corresponding to the maximum signal between 38° and 40° are observed in other samples that are dried in other apparatuses (e.g., an oven) to the same maximum temperatures then cooled to room temperature.

This example illustrates that a structure change in the final catalyst when heated to higher temperatures. Without being limited by theory, it is believed that 160° C. and higher drying after alkali metal promoter impregnation is needed to see this structure change.

Therefore, the present invention is well adapted to attain the ends and advantages mentioned as well as those that are inherent therein. The particular examples and configurations disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative examples disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

1. A method comprising:
impregnating a porous support with a water-insoluble gold compound and a water-insoluble palladium compound to yield a precipitated support by precipitating a water-soluble gold compound and a water-soluble palladium compound in the presence of the porous support;
washing the precipitated support;
reducing the water-insoluble gold compound and the water-insoluble palladium compound on the precipitated support to yield a metal-impregnated support;
impregnating the metal-impregnated support with an alkali metal promoter to yield a metal/promoter-impregnated support; and
drying the metal/promoter-impregnated support at 160° C. or greater to yield a catalyst.

2. The method of claim 1, wherein the impregnating of the porous support is performed in multiple steps comprising:
impregnating the porous support with a first water-soluble compound that is the water-insoluble gold compound or the water-insoluble palladium compound;
precipitating the first water-soluble compound in the presence of the porous support;
impregnating the porous support with a second water-soluble compound that is the water-insoluble gold compound or the water-insoluble palladium compound, wherein the first and second water-soluble compounds are different; and
precipitating the second water-soluble compound in the presence of the porous support.

3. The method of claim 1, wherein a molar ratio of the gold to the palladium in the catalyst is about 0.01:1 to about 0.7:1.

4. The method of claim 1, wherein an alkali metal of the alkali metal promoter is present at about 0.1 wt % to about 10 wt % of the catalyst on a dry basis.

5. The method of claim 1, wherein the alkali metal promoter is selected from the group consisting of a sodium salt, a potassium salt, or a cesium salt of formic acid, acetic acid, propionic acid, butyric acid, and any combination thereof.

6. The method of claim 1, wherein the reducing is in a gas phase comprising hydrogen and/or a hydrocarbon.

7. The method of claim 6, wherein the hydrocarbon is ethylene.

8. The method of claim 6, wherein the gas phase further comprises an inert carrier gas.

9. The method of claim 6, wherein the reducing is at about 50° C. to about 250° C. for about 1 hour to about 24 hours.

10. The method of claim 1, wherein the reducing is performed in a liquid phase using hydrazine hydrate.

11. The method of claim 9, wherein the reducing is at about 20° C. to about 50° C. for about 1 hour to about 24 hours.

12. The method of claim 1, wherein the drying of the metal/promoter-impregnated support is at about 160° C. to about 250° C.

13. The method of claim 12, wherein the drying of the metal/promoter-impregnated support is for about 10 minutes to about 1 day.

14. The method of claim 1, wherein the drying of the metal/promoter-impregnated support is in the presence of gas comprising nitrogen, argon, and/or air.

15. The method of claim 1, wherein the catalyst has a 20 value for a peak x-ray diffraction intensity between 38° and 40° of about 38.6° to about 39.2°.

16. A catalyst comprising:
(a) gold, palladium, and/or gold-palladium alloy, (b) an alkali metal promoter, and (c) a porous support, wherein the catalyst has a 20 value for a peak x-ray diffraction intensity between 38° and 40° of about 38.6° to about 39.2°.

17. The catalyst of claim 16, wherein a molar ratio of the gold to the palladium, cumulatively as elemental metals and alloy, in the catalyst is about 0.01:1 to about 0.7:1.

18. The catalyst of claim 16, wherein an alkali metal of the alkali metal promoter is present at about 0.1 wt % to about 10 wt % of the catalyst on a dry basis.

19. The catalyst of claim 16, wherein the alkali metal promoter is selected from the group consisting of a sodium salt, a potassium salt, or a cesium salt of formic acid, acetic acid, propionic acid, butyric acid, and any combination thereof.

20. The catalyst of claim 16, wherein the porous support is selected from the group consisting of: silica, alumina, aluminum silicates, titania, zirconia, spinels, carbon, and any combination thereof.
